(19) United States
(12) Patent Application Publication
(10) Pub. No.: US 2010/0323397 A1
(43) Pub. Date: Dec. 23, 2010

(54) MODIFIED VIRD2 PROTEIN AND ITS USE IN IMPROVED GENE TRANSFER

(75) Inventors: Brian Reavy, Angus (GB); Mikhail Talianski, Dundee (GB); Sang Hyon Kim, Dundee (GB); Andrey Borisowich Vartapetian, Moscow (RU); Nina Valentinovna Chichkova, Moscow (RU); Svetlana Bagirova, Glasgow (GB)

Correspondence Address: PEPPER HAMILTON LLP, ONE MELLON CENTER, 50TH FLOOR, 500 GRANT STREET, PITTSBURGH, PA, 15219, US

(73) Assignee: SCOTTISH CROP RESEARCH INSTITUTE, Dundee (GB)

(21) Appl. No.: 12/300,481

(22) PCT Filed: May 10, 2007
(86) PCT No.: PCT/GB07/01723

§ 371 Date: Jun. 30, 2010

(30) Foreign Application Priority Data
May 12, 2006 (GB) 0609420.5

(51) Int. Cl. C12P 21/00; C12N 15/82; C12N 15/63; C07K 14/195; C07H 21/00; C12N 15/85

(52) U.S. Cl. 435/69.1; 435/469; 435/320.1; 530/350; 536/23.7; 435/455

(57) ABSTRACT

There is provided a method for Agrobacterium-mediated gene transformation of a host cell. The method uses an Agrobacterium which expresses a VirD2 protein resistant to cleavage by a caspase. The VirD2 protein is modified by replacement of an aspartic acid residue with an alternative amino acid at one or more of the caspase cleavage sites within the protein amino acid sequence. This method improves the low efficiency of Agrobacterium-mediated gene transfer in certain plant cells and in animal cells caused by caspase cleavage of the VirD2 protein. The modified VirD2 protein, a gene for its expression and a method of producing a protein of interest by Agrobacterium-mediated transformation of a host cell are also described.

The present invention provides an improved methodology for gene transfer. In particular the present invention provides an improved method for gene transfer mediated by Agrobacterium.

Agrobacterium tumefaciens, a soil plant pathogenic bacterium, is able to mediate gene transfer in dicotyledonous plants and is now very widely used for this purpose in plant genetic engineering. A. tumefaciens naturally infects wounds of dicotyledonous plants leading to the development of crown gall tumour. It has been recognised (see Nester et al., 1984, Annual Review of Plant Physiology 35:387-413; Binns and Thomashaw 1988, Annual Review of Microbiology 42:575-606) that this disease development was due to the ability of A. tumefaciens to transfer a DNA segment, termed T-DNA, from the tumour inducing (Ti) plasmid into the nucleus of infected plant cells where it was then stably integrated into the host genome and transcribed.

De la Riva et al., (in EJB, Vol. 1, No. 3, December 1998: 118-133) review of the use of A. tumefaciens in plant transformation and its putative mechanism. De la Riva et al., state that 25-bp direct repeats flank the T-DNA fragment and these act as a cis element signal for transfer, and that the process of T-DNA transfer is mediated in part by the Ti plasmid virulence region (Vir genes). The 30 kb virulence region is organised in six operons that are essential for T-DNA transfer (VirA, VirB, VirD and VirG) or for increasing the efficiency of transfer (VirC and VirE).

Studies on the T-DNA transfer process (reviewed by Torisky et al., 1997, Plant Cell Reports 17:102-108) confirm that the crown gall tumour formation results from transfer and integration of T-DNA into the plant cells, and the subsequent expression of the T-DNA genes. The T-DNA genes themselves do not also mediate transfer. Finally, any foreign DNA placed between the T-DNA borders can be transferred to plant cells, irrespective of the origin of the foreign DNA. Elucidation of these criteria has allowed Agrobacterium-mediated gene transfer to be used widely for dicotyledonous plants, even plants outside the normal host range of A. tumefaciens. The advantages of Agrobacterium-mediated gene transfer technology compared with other (direct) methods of gene transfer is the defined insertion of a discrete segment of DNA into the recipient genome avoiding integration of multiple transgene copies which frequently leads to gene silencing and inefficient gene expression.

More recently, modified methodologies have been developed for monocotyledonous plants (see de la Riva et al., 1998 supra) but in general the efficiency of such transformation in economically important monocotyledonous plants, such as cereals, or in animal cells is extremely low. One of the major factors affecting the efficiency of Agrobacterium-mediated transformation of plants is a strong necrotic hypersensitive response (HR); a type of plant programmed cell death (PCD) to Agrobacterium.

Programmed cell death (PCD), or apoptosis, is a fundamentally important process that maintains the integrity and homeostasis of organisms, regulates their growth, development and responses to pathogen attacks and abiotic stresses. Caspases (cysteinyl aspartate-specific proteinases) have been identified as essential elements in the cell-suicide machinery, and have been shown to play a critical role in mammalian PCD. Caspases are responsible for the proteolysis of key proteins that are known to be selectively cleaved at the onset of apoptosis. However, in spite of the striking similarities between PCD pathways in animals and plants, the case for any existence of caspases in plants has been controversial. Although some specific inhibitors of animal caspases have been shown to affect development of PCD in plants, no direct homologues of animal caspase genes have been identified in plants (Chichkova et al., (2004), Plant Cell, 16, 157-171).

Chichkova et al., 2004 demonstrated that a capase-like protein is activated and causes PCD in tobacco during N-gene mediated HR triggered by Tobacco mosaic virus (TMV).

Further, the Agrobacterium tumefaciens VirD2 protein is specifically cleaved at two sites (TATD and GEQD) by human caspase-3. VirD2 was used as a target for the detection of a putative caspase-like protein in tobacco. In tobacco leaves, specific proteolytic processing of the ectopically produced VirD2 derivatives at these sites was found to occur early in the HR triggered by TMV. A proteolytic activity capable of specifically cleaving the model substrate at TATD was partially purified from these leaves. A tetrapeptide designed and synthesized on the basis of the elucidated plant caspase cleavage site prevented fragmentation of the substrate protein in vitro and counteracted TMV-triggered HR in vivo. Thus, the plant enzyme investigated by Chichkova et al., 2004, is suggestive of a novel functional analogue of animal caspases.

We have since purified the capase-like protein described by Chichkova et al., 2004, supra. Specifically, tobacco cell extracts were fractionated by DEAE anion-exchange chromatography, and the caspase was further purified using a biotinylated derivative of the TATD-CHO tetrapeptide aldehyde as an affinity ligand. The enzyme-inhibitor complex was collected with streptavidin-agarose, eluted with biotin, resolved by SDS-PAGE, and a protein band corresponding to a molecular mass of 82 kDa was visualized by silver staining. To identify this protein, the corresponding band was cut from the gel, digested with trypsin and analyzed by matrix-assisted laser desorption time-of-flight (MALDI-TOF) mass spectrometry (MS) which detected several tryptic peptides matching an 82 kDa putative subtilisin-like (serine) protease (PSLP) in A. thaliana (accession number gi:18400323; NP 566483).

Recently, two other subtilisin-like serine proteases from Avena sativa were shown to exhibit caspase specificity (Coffeen and Wolpert (2004) Plant Cell, 16, 857-873). However in contrast to these others, the protease isolated in our work (Chichkova et al., 2004) was not inhibited by serine protease inhibitors such as chymostatin but was inhibited by the cysteinyl protease inhibitor mercuric chloride. Moreover, again in contrast to the A. sativa caspase-like serine proteases, our protease was not inhibited by VAD nor by DEVD based peptide inhibitors.

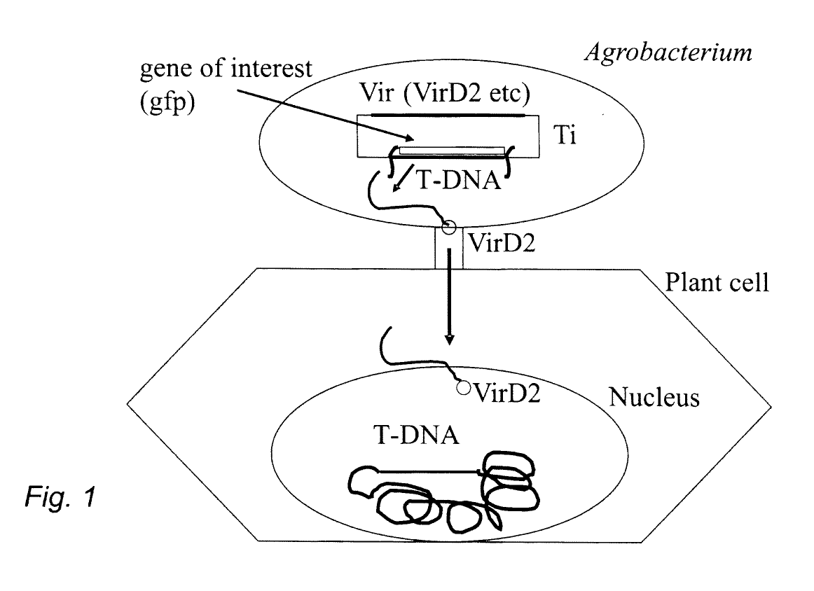

As mentioned above, Agrobacterium-mediated gene transfer is based on its ability to transfer and randomly integrate into the genome of plant a specific fragment of its tumour-inducing plasmid (Ti) known as the transferred DNA (T-DNA). In nature, the transferred genetic information is essential for pathogenesis. However, as all the genes required for production and transfer of T-DNA reside outside of the T-DNA, its pathogenic sequences can be replaced by a gene(s) of interest, thus making Agrobacterium a powerful tool of genetic engineering. VirD2 protein was shown to have a key role in the nuclear uptake and genomic integration of T-DNA in plants (FIG. 1).

We have now shown that Agrobacterium-mediated transformation of plants activate plant caspases (PSLP) which cleave the VirD2 protein thereby affecting its function. This understanding has led to the realisation that the low efficiency of Agrobacterium-mediated transformation in certain plant cells and in animal cells is a consequence of caspase cleavage of the VirD2 protein.

The present invention is thus concerned with improving the efficiency of Agrobacterium-mediated gene transfer by reducing cleavage of the VirD2 protein by plant (PSLP) and/or animal caspases. More surprisingly we have also found that stable transformation of the host cells was significantly increased.

It is moreover now recognised that other bacteria outside the Agrobacterium genus can also be modified to mediate gene transfer in a number of diverse plants, such as Rhizobium, Sinorhizobium and Mesorbium strains (see Broothaerts et al., (2005) Nature, 433, pages 629-632). Such bacteria able to mediate gene transfer are included within the term “Agrobacteria” as used herein, and are able to conduct “Agrobacterium-mediated gene transfer”, as used herein.

As defined herein the term “protein” includes any peptide, polypeptide or protein irrespective of molecular size.

The present invention thus provides a VirD2 protein modified so that it is resistant to cleavage by caspases. The caspases can be an animal caspase or a plant caspase (also termed a subtilisin-like serine protease, PSLP) or animal homologues of plant caspases. In one embodiment, at least one of the cleavage sites TATD, GEQD, PVTD, VNLD, ASLD and DEVD (SEQ ID Nos 1 to 6, respectively) is modified by replacement of the D residue with an alternative amino acid but the invention excludes a VirD2 protein wherein the only modification is of the cleavage sites TATD and/or GEQD. In one embodiment at least one of the cleavage sites PVTD, VNLD, ASLD and DEVD is modified, for example by replacement of the D residue with an alternative amino acid. Suitable alternative amino acids include glycine, alanine, valine, leucine, isoleucine, proline, phenylalanine, methionine, tryptophan, cysteine, glutamic acid, lysine, arginine, histidine, asparagine, glutamine, serine, threonine and tyrosine. Conveniently alanine, asparagine or glutamic acid may be used.

In one embodiment the modified VirD2 protein also has at least one (optionally both) of the cleavage sites TATD or GEQD modified as described above.

In one embodiment (for example suitable for the C58 strain of Agrobacterium tumefaciens), at least one of the cleavage site D residues (positions 371 and 400) in the VirD2 protein are modified. One suitable replacement amino acid is alanine, but one of ordinary skill in the art would be aware that other amino acids could also be used. Asparagine, Asn (N) or glutamic acid, Glu (E) may be suitable in certain circumstances.

In this embodiment, the following portion of VirD2 protein:

(SEQ ID No. 7)
[361 vgpqanageq dgssgplvrq agtsrpsppt attrastatd
slsatahlqq rrgvlskrpr 420].

in which the D residues of the cleavage sites (shown underlined) were replaced with alanine to give the following modified portion:

(SEQ ID No. 8)
[361 vgpqanageq agssgplvrq agtsrpsppt attrastata
slsatahlqq rrgvlskrpr 420].

In another embodiment, the modified VirD2 protein has the cleavage site PVTD, wherein for example the cleavage site D residue can be modified.

One suitable replacement amino acid is alanine, but one of ordinary skill in the art would be aware that other amino acids could also be used. Asparagine, Asn (N) or glutamic acid, Glu (E) may be suitable in certain circumstances.

As an example, the cleavage site present in the VirD2 protein of the octopine strain of Agrobacterium tumefaciens at (322)PVTD(325) could be modified to (322)PVTA(325) (SEQ ID No. 9).

In another embodiment, the modified VirD2 protein has at least one of the cleavage sites VNSL or ASLD, wherein for example the cleavage site D residue can be modified. One suitable replacement amino acid is alanine, but one of ordinary skill in the art would be aware that other amino acids could also be used. Asparagine, Asn (N) or glutamic acid, Glu (E) may be suitable in certain circumstances.

The modified VirD2 protein of the present invention can have more than a single cleavage site modified as described above. Thus, the modified VirD2 protein can have two, three, four or more cleavage sites modified. In one embodiment the modified VirD2 protein has at least one of the cleavage sites PVTD, VNLD, ASLD and DEVD modified by replacement of the D residue with an alternative amino acid, and also has one or both of the cleavage sites TATD and/or GEQD modified by replacement of the D residue with an alternative amino acid.

The present invention also provides a gene encoding a modified VirD2 protein as described above. The gene can be based upon a wild type allele but wherein the codon for aspartic acid (Asp, D) in the required location is not present but instead includes a codon for an alternative amino acid in that location. The codons which normally encode aspartic acid are GAC and GAU. Single base substitutions can alter such codons to AAC and AAU (to encode asparagine; to CAC and CAU (to encode histidine); to AUC and AUU (to encode tyrosine); to GUU and GUC (to encode valine); to GCA and GCC (to encode alanine); to GGU and GGC (to encode glycine); or to GAA and GAG (to encode glutamic acid). Further nucleotide substitutions can be used to extend the range of alternative amino acids. Techniques for site directed mutagenesis of a specific codon are well known with the art.

The present invention also provides modified strains of Agrobacterium (especially A. tumefaciens) which express a modified VirD2 protein as described above. In this embodiment the Agrobacterium sp comprises a gene encoding the modified VirD2 protein. Techniques to produce the required modification to the VirD2 gene are well known in the art.

The modified Agrobacterium can be used as a vector. The modified Agrobacterium can also include a gene encoding a protein of interest with the T-DNA. The protein of interest can be any protein (which term includes both peptides and polypeptides) the expression of which is advantageous, either providing a desired phenotype to the host cell or producing a protein for harvest (e.g. an enzyme, pharmaceutical, hormone or other commercially significant protein).

In an alternative embodiment, the VirD2 protein can be protected from degradation by caspases, by inducing caspase gene knock-down or gene silencing in the target host. Where the target host is a plant, plant caspase (PSLP) gene knockdown or virus induced PSLP gene silencing (VIGS) can be used.

In a further aspect, the present invention provides a method of Agrobacterium-mediated gene transformation of a host cell, said Agrobacterium having a VirD2 protein, wherein caspase-mediated degradation of the VirD2 protein is reduced, relative to that of the wild type VirD2 protein in a wild type host cell.

In the method claimed a gene encoding a protein of interest is inserted into the T-DNA of the Agrobacteria using known techniques and, following infection of the host cell by the Agrobacteria, is stably integrated into the genome of the host cell. We have found that successful transformation of the T-DNA is increased where the VirD2 protein is modified to be capase-resistant.

The present invention further provides a method of producing a protein of interest, said method comprising:

i) providing an Agrobacterium vector encoding a VirD2 protein modified to have at least one aspartic acid (D) residue replaced by an alternative amino acid, and further comprising a gene encoding a protein of interest within the T-DNA thereof;
ii) infecting a host cell with said Agrobacterium under conditions suitable to allow stable integration of the gene encoding the protein of interest into the genome of said host cell; and
iii) cultivating said host cell or progeny thereof under conditions suitable to allow expression of said protein of interest.

The host cell may be cultivated in vivo or may form part of a larger organism (plant or animal). Where the host cell is part of a plant, the progeny of the plant (especially the vegetative progeny) are included within the present invention.

In a further aspect, the present invention provides a method of gene transformation in a host cell, whereby said transformation is mediated by Agrobacterium having a VirD2 protein resistant to degradation by the host cell. The VirD2 protein can be a modified VirD2 protein as described above. The host cell can be a plant cell or animal cell (e.g. mammalian, insect or other animal cell). The plant host cell can be a monocotyledonous or dicotyledonous plant cell. Host plant cells of commercial importance include cereals (e.g. maize, barley), oil seed rape, soybean and potato, and animal cells of commercial importance include mouse C127 and L929, Chinese hamster ovary (CHO), bovine hamster kidney (BHK), Drosphila melanogaster (DS2) and Spodoptera frugiperda (Sf9).

In one embodiment, the Agrobacterium VirD2 protein has been modified to be resistant to host cell caspase-mediated degradation, for example as described above.

In any such embodiment, the caspase can be an animal cell caspase (e.g. from an insect cell, mammalian cell or other animal cell) or can be a plant caspase (or PSLP).

In one embodiment, the host cell has been modified to reduce caspase-mediated degradation of the Agrobacterium VirD2 protein.

The host cell can be a plant cell or animal cell (e.g. mammalian, insect or other animal cell). The plant host cell can be a monocotyledonous or dicotyledonous plant cell. Host plant cells of commercial importance include cereals (e.g. maize, barley), oil seed rape, soybean and potato, and animal cells of commercial importance include mouse C127 and L929, Chinese hamster ovary (CHO), bovine hamster kidney (BHK), Drosphila melanogaster (DS2) and Spodoptera frugiperda (Sf9).

The present invention will now be further described by reference to the following, non-limiting, examples and to the figures in which:

FIG. 1 is a simplified representative of T-DNA transfer, showing the key role of the VirD2 protein for the transport and integration of T-DNA into the host cell (depicted as a plant cell).

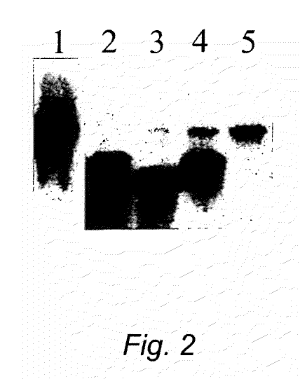

FIG. 2 Western (immunoblotting) analysis indicating degradation of wild type (WT) VirD2 protein by plant caspase (lane 2), partial resistance of VirD2 single mutants (D371A and D400A) (lanes 3 and 4) and complete resistance of VirD2 double mutant (D371, 400A) (lane 5) to plant caspase. Lane 1 represents untreated wild type (WT) VirD2 protein.

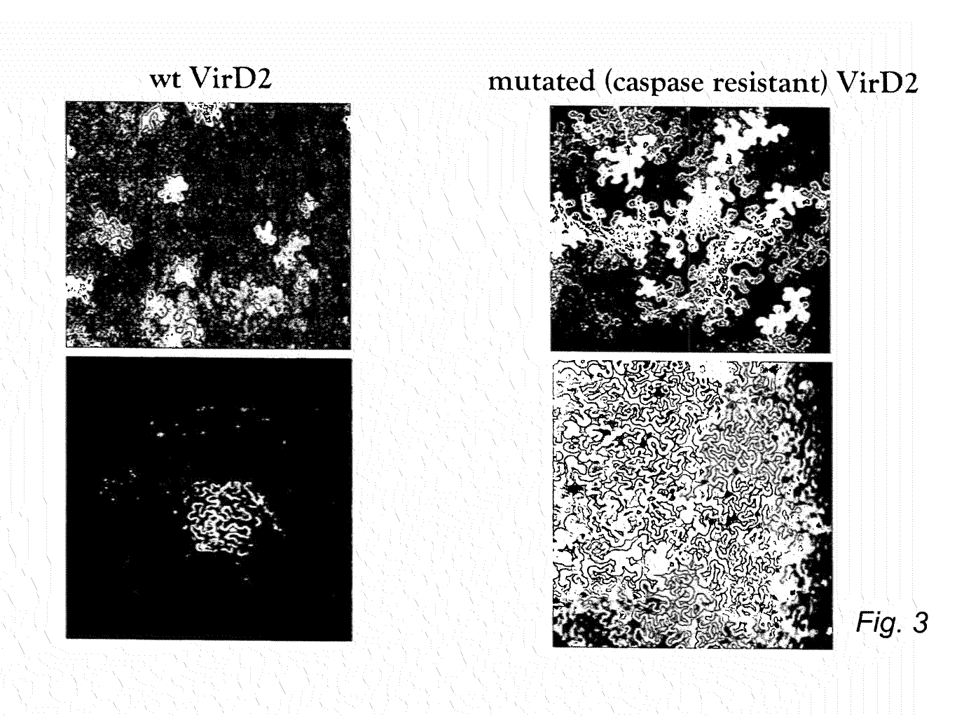

FIG. 3 shows Agrobacterium-mediated expression of GFP achieved using modified VirD2 protein in Nicotiana benthamiana plants, compared to transformation using wild type (WT) VirD2.

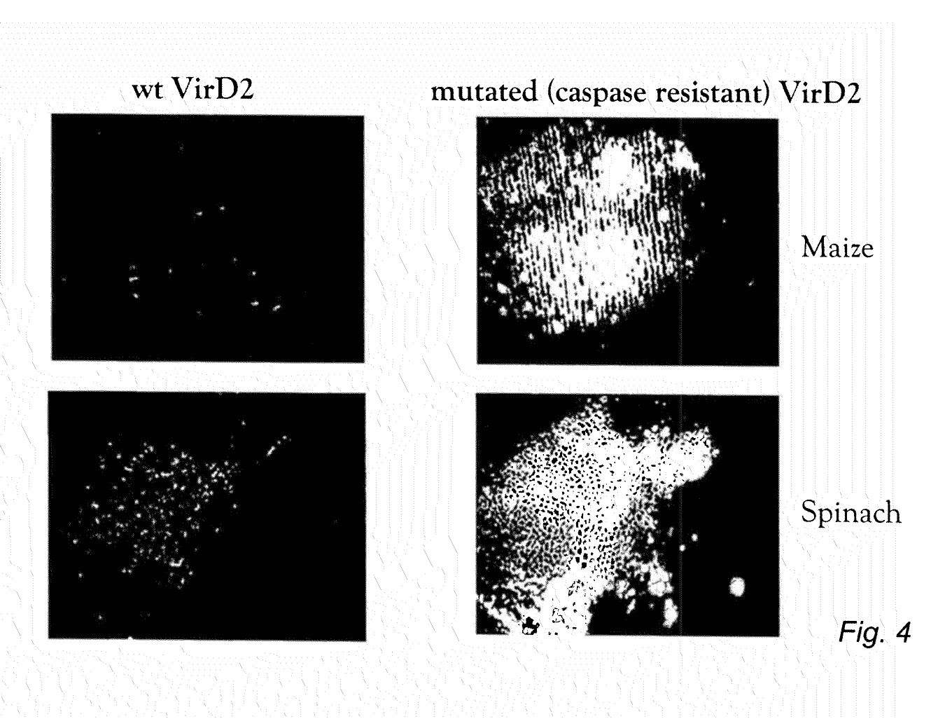

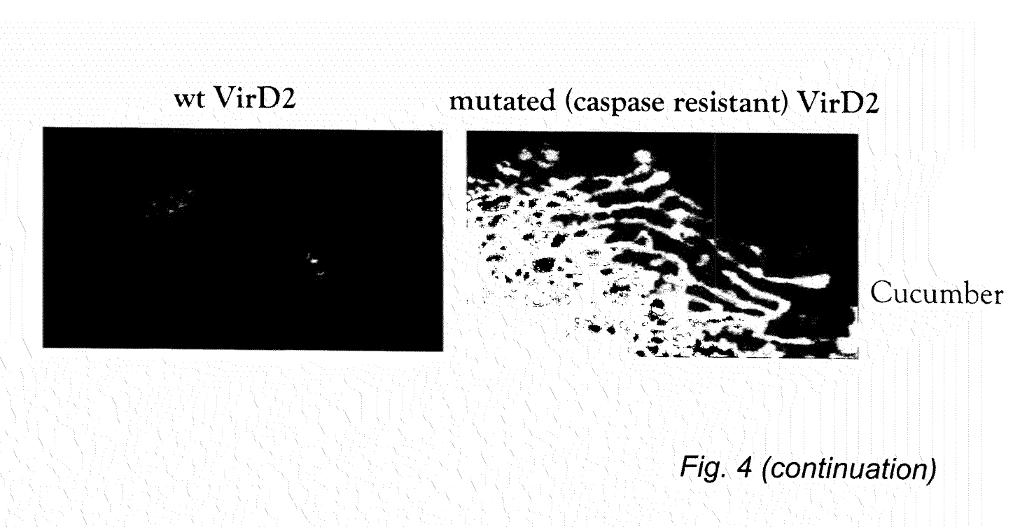

FIG. 4 shows Agrobacterium-mediated expression of GFP achieved using modified VirD2 protein in maize, spinach and cucumber plants, compared to transformation using wild type (wt) VirD2.

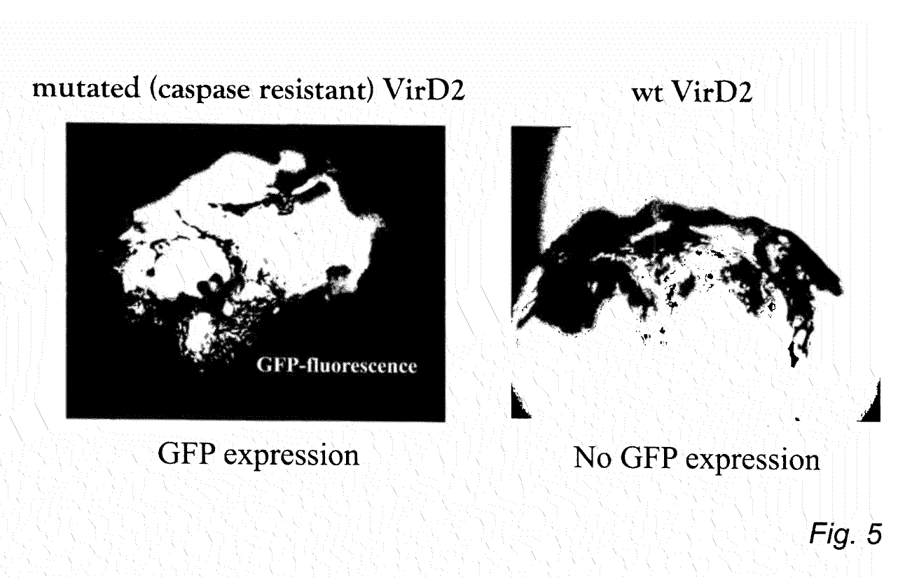

FIG. 5 shows Agrobacterium-mediated expression of GFP achieved using modified VirD2 protein in oil seed rape cotyledons, compared to transformation using wild type (wt) VirD2.

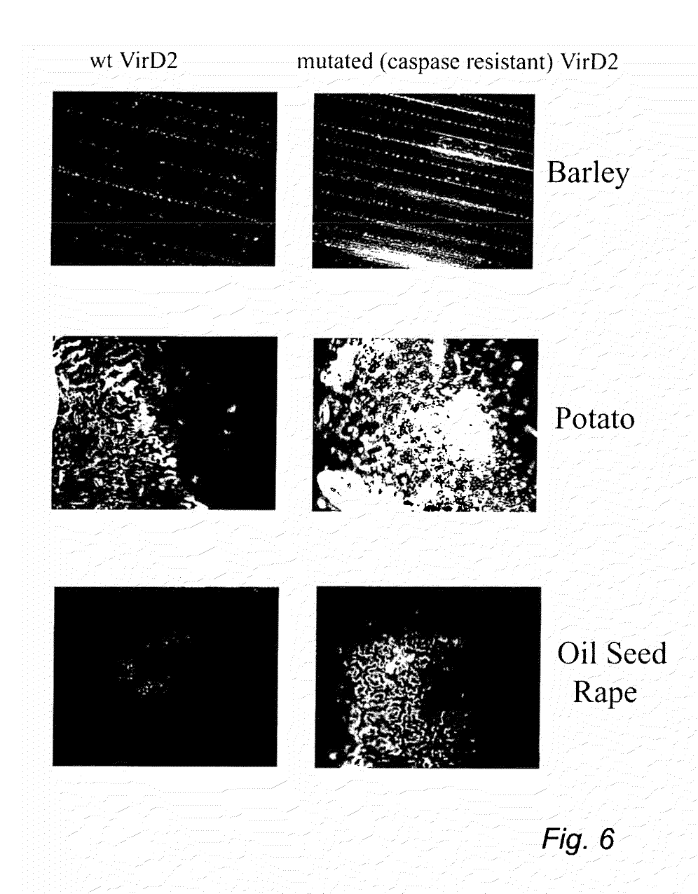

FIG. 6 shows Agrobacterium-mediated expression of GFP achieved using modified VirD2 protein in barley, potato and oil seed rape plants, compared to transformation using wild type (wt) VirD2.

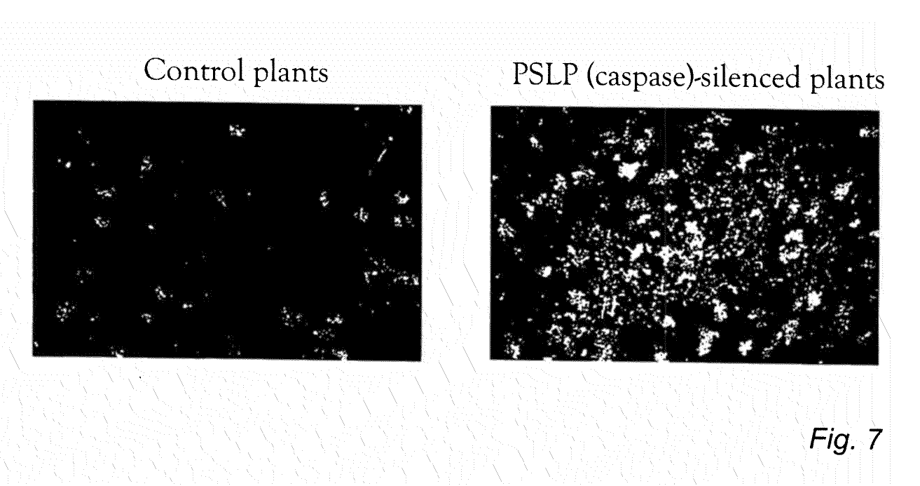

FIG. 7 shows Agrobacterium-mediated expression of GFP in Nicotiana benthamiana silenced for the PSLP gene or not (control).

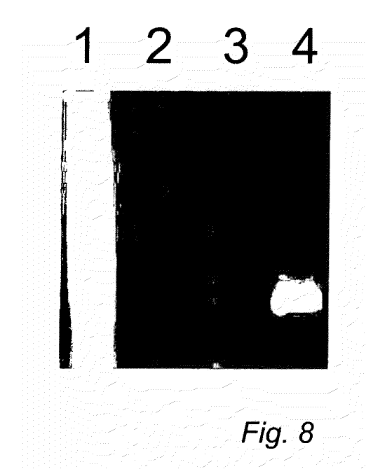

FIG. 8 shows PCR examination of Drosophila cell genomic DNA for the presence of GFP DNA from: 1. Untreated cells, 2. Cells co-cultivated with Agrobacteria containing wild type VirD2, 3. Cells co-cultivated with Agrobacteria containing mutant VirD2. Lane 4 shows GFP DNA amplified from a plasmid control.

### EXAMPLES

### Example 1

### Mutation of VirD2 Protein

The protein-encoding portion of virD2 cDNA [Agrobacterium tumefaciens str. C58 (a nopaline-type Ti plasmid strain); accession No. NP 536300] in pQE13 vector (Qiagen) was used for this work. The virD2 cDNA was then excised from this plasmid as a 100-bp SauIIIA-PstI and a 1240-bp PstI-XbaI DNA fragments and inserted between the BamHI and XbaI sites of pUC19 to produce pUC19NirD2. Elimination of the Ec113611-Bsp1201 fragment from pUC19NirD2, filling-in with the Klenow fragment, and self-ligation of the rest of the plasmid produced pUC19/VirD2Ct.

Mutations were introduced in the virD2 sequence by PCR on pUC19NirD2Ct with the mutagenic primers 5′-CTACTGCCAgCCTGTTCGC-3′ [for VirD2Ct(D371A)] (SEQ ID No. 10), 5′-GACAATGAAgCGGTTGCG-3′ [for VirD2Ct(D400A)] (SEQ ID No. 11) (lower case letters indicate the nucleotide substitutions) and the pUC19 direct and reverse sequencing primers. The PCR products were digested with EcoRI and BamHI, ligated into pUC19 and sequenced to check for the absence of unwanted mutations. The double (D371,400A) mutant was constructed by DNA shuffling using a DdeI site located between the corresponding mutations to produce pUC19/VirD2Ct(D371,400A).

To convert the octopine-type Ti plasmid-encoded VirD2 located in pAVD43 plasmid into C58-encoded VirD2, the PstI-XbaI DNA fragment from pUC19/VirD2 was first inserted into similarly cleaved pUC19 to produce pUC19/Pst-Xba. Then a 1250-bp DNA fragment was excised from pUC19/Pst-Xba with EcoRI (partial digestion)+Pstl and used to substitute the VirD2-encoding PstI-EcoRI fragment of pAVD43 (Rossi et al. (1993) Mol Gen Genet, 239, 345-353), the resultant plasmid being named pAVD43-058 wt. The presence of an internal EcoRI site in the VirD2 cDNA of C58 was employed to confirm the rearrangement.

To obtain pAVD43-058(D371,400A) encoding the plant caspase resistant VirD2(D371,400A) mutant, the Pstl-Xbal DNA fragment in pUC19/Pst-Xba was substituted with the analogous DNA fragment from pUC19NirD2Ct(D371,400A). Elimination of a HinfI site due to one of the D/A mutations was employed to confirm successful substitution had occurred. The VirD2(D371,400A)-encoding cDNA fragment was then transferred to pAVD43 (as described above for the wt VirD2 version) to give pAVD43-VirD2(D371,400A).

Two different strategies were used to express mutated VirD2(D371,400A)-protein. In the first (replacement) strategy we inserted the VirD2 constructs [pAVD43-058 wt/control and pAVD43-VirD2 (D371,400A)] generated above into the GV3101 (pPM6000K) strain of Agrobacterium carrying the VirD2 deletion by conjugation as described by Rossi et al. (1993) Mol Gen Genet, 239, 345-353. In the second (complementation) strategy we inserted pAVD43-058 wt/control and pAVD43-VirD2(D371,400A) constructs to normal Agrobacterium strains carrying their own VirD2 genes. Both approaches give similar results. To monitor efficiency of gene transfer, modified Agrobacterium strains were electroporated with additional plasmids expressing green fluorescent protein under control of 35S cauliflower mosaic virus (CaMV) promoter.

Final Agrobacterium strains were propagated and used in agroinfiltration assay (transient expression assays).

FIG. 3 clearly shows that modified VirD2 protein resistant to plant caspase (in the Agrobacterium strain carrying pAVD43-VirD2(D371,400A)) significantly promotes expression of ectopic G FP protein in Nicotiana benthamiana (model) plants; the number of GFP-expressing cells is increased significantly.

Similar results were also obtained with both monocotyledoneous (cereal, maize; see FIG. 4) and dicotyledenous (spinach and cucumber; see FIG. 5, barley, potato and oil seed rape; see FIG. 6) crops. It is important to note that not only the number of fluorescent cells, but also the intensity of fluorescence is significantly increased suggesting that not only gene transfer but also its integration into plant genomes (stable transformation) is stimulated. Resistance of mutated VirD2 [VirD2(D371,400A)] protein to plant caspase fragmentation has been confirmed (FIG. 2b)

### Example 2

### Stable Expression

We tested gene (GFP) expression following stable transformation of oilseed rape cotyledons using the method described for the Brassica napus system by M. M. Moloney et al. (1989) Plant Cell Reports 8: 238-242. Surface sterilised seeds were sown on germination medium. After 4 days cotyledons were excised from the seedlings. They were inoculated by dipping into an Agrobacterium solution. The cotyledons were then returned to co-cultivation plates. Inoculated cotyledons were regularly transferred to fresh medium. The cut ends initiated callus after the first couple of weeks. The callus obtained using Agrobacterium expressing mutated VirD2 protein resistant to plant caspase [VirD2(D371,400A)] displayed strong green fluorescence whereas that obtained using Agrobacterium expressing wt VirD2 protein susceptible to plant caspase (VirD2-058 wt) did not (FIG. 5); sometimes some weak fluorescence in this case developed after a delay of 2-3 weeks. These results clearly show that modification (mutagenesis) of two potential plant (PSLP) caspase cleavage sites TATD and GEQD in the VirD2 protein to make the protein resistant to caspases significantly increases efficiency of gene transfer and integration in the plant genome.

### Example 3

### Plant Capase (PSLP) Gene Knock-Down

To isolate a DNA fragment corresponding to the PSLP gene, the first strand cDNA was generated using mRNA purified from tobacco (N. tabacum cv. Samsun NN) and oligo-dT primer. RT-PCR was done using the first strand cDNA and primers TC178 (5′-ATC ATT GGC GCT CGT TAC TTC-3′) (SEQ ID No. 12) and TC243 (5′-CTT GTA CAT AGC CAC ATG AGC-3′) (SEQ ID No. 13), which correspond to peptides 178-IIGARYFNK (SEQ ID No. 14) and AHVAMYK-243 (SEQ ID No.15), respectively identified in the tobacco PSLP. The PCR product of 210 by in size was cloned into pGEM-T vector (Promega Co.), and two clones with different orientation, designated pGEM(178-243) and pGEM(243-178), were obtained, and sequenced. Three RNAi fragments for sense, antisense and hairpin were then produced:

1) The sense fragment was synthesized by PCR using TC243attB1(5′-GGGG ACA AGT TTG TAC AAA AAA GCA GGC T CTT GTA CAT AGC CAC ATG AGC-3′) (SEQ ID No. 16) and TC178attB2 (5′-GGG GAC CAC TTT GTA CAA GAA AGC TGG GT ATC ATT GGC GCT CGT TAC TTC-3′) (SEQ ID No. 17), and pGEM(178-243) as template. The sequence of the fragment was determined:

(SEQ ID No. 18)
ATCATTGGCGCTCGTTACTTCAATAAAGGCCTACTTGCCAACAATCCAAA
TCTTAACATTTCAATGAATTCTGCTAGAGATACCGATGGACATGGAACTC
ACACTTCTTCTACAGCTGCGGGAAGTTATGTCGAGGGTGCATCTTATTTT
GGCTATGCCACTGGCACTGCTATAGGCATAGCACCAAAGGCTCATGTGGC
TATGTACAAG.

2) The antisense sequence was generated by TC178attB1 (5′-GGGG ACA AGT TTG TAC AAA AAA GCA GGC T ATC ATT GGC GCT CGT TAC TTC-3′) (SEQ ID No. 19) and TC243attB2 (5′-GGG GAC CAC TTT GTA CAA GAA AGC TGG GT CTT GTA CAT AGC CAC ATG AGC-3′) (SEQ ID No. 20), and pGEM(178-243).
3) For the hairpin construct, pGEM(178-243) and pGEM(243-178) were digested with Ncol, ligated with T4 DNA ligase and used as template for TC178attB1 and TC178attB2 primers.

The PCR products were inserted into pDONR207 vector (Invitrogen) by BP reaction by BP clonase (Invitrogen), resulting in pENTR(178-243), pENTR(243-178) and pENTR(178-178) for sense, antisense and hairpin constructs, respectively. They were subjected to LR reaction (Invitrogen) together with pBIN(TRV-RNA2)attR (a binary vector for plant transformation containing TRV RNA2 amplicon under 35S promoter to generate pBIN[TRV-RNA2(178-243)], pBIN[TRV-RNA2(243-278)] and pBIN[TRV-RNA2(178-178)] for antisense, sense and hairpin constructs, respectively, and transferred to Agrobacterium tumefaciens strain GV3101. For silencing of PSLP gene, Nicotiana benthamiana plants were infiltrated with pBIN[TRV-RNA2(178-243)], pBIN[TRV-RNA2(243-278)] or pBIN[TRV-RNA2(178-178)] into underneath of leaf. VIGS developed approximately ten days post-inoculation.

To monitor the effect of VIGS on Agrobacterium-mediated gene transfer, the silenced and non-silenced (control) N. benthamiana plants were challenged with Agrobacterium strain carrying a binary vector expressing alpha GFP under control of the 35S promoter. Three days post challenge inoculation, the inoculated leaves were detached for analysis under Bio-Rad MRC confocal laser scanning microscope. FIG. 7 shows that expression of GFP in PSLP silenced plants is at least five-fold greater than in non-silenced plants.

Thus prior silencing of PSLP in plants can lead to significant increase in the efficiency of Agrobacterium-mediated gene transfer which potentially may be used for transient expression systems and for stable plant transformation.

### Example 4

### Agrobacterium-medicated Gene Transfer to Animal Cells

Taking into account that the VirD2 protein is also a target for animal caspases approaches for protection of the VirD2 protein from plant caspases as described above (modification of VirD2 protein to make it resistant to caspases and/or silencing of caspase gene) can also be applied to non-plant (animal/mammalian/insect) cells thereby extending the applicability of Agrobacterium-mediated gene transfer to a virtually unlimited range of plant and non-plant recipient systems by protection of VirD2 protein from plant (PSLP) and animal caspases.

These methods can be used for example for transformation of mammalian cells for production of pharmaceuticals and industrial proteins. Existing non-Agrobacterium-based (direct) methods of gene transfer in mammalian cells often result in integration of multiple transgene copies which frequently leads to gene silencing and inefficient gene expression.

We demonstrated efficient gene transfer to animal cells using Agrobacteria containing the mutant VirD2 in the following way. Drosophila DS2 cells were grown in Schneider's Drosophila medium containing 10% foetal bovine serum (Invitrogen). 5 ml cultures of Drosophila DS2 cells were seeded at approximately 10⁶ cells in 25 cm² tissue culture flasks (Greiner). The cells were grown at 28° C. for 24 hours before inoculation with Agrobacterium tumefaciens containing wild type VirD2 and a plasmid encoding green fluorescent protein (GFP), or with this strain containing mutant VirD2 and a plasmid encoding GFP. Agrobacteria cultures were grown at 28° C. for 16 hours in LB medium containing 50 μg/ml Kanamycin and 100 μg/ml spectinomycin in 5 ml cultures shaken at 250 rpm. 100 μl of these cultures were then used to inoculate 5 ml cultures of LB containing 100 μM A acetosyringone to induce vir gene expression and grown with shaking for 5 hours. Drosophila cell cultures were inoculated with 100 μl of either culture and grown for 48 hours at 28° C. in medium containing 0.1 μM acetosyringone. After this co-incubation cells were washed twice with medium and grown for 3 weeks in medium containing 0.2 mM cefotaxime to remove the agrobacteria with medium being changed every 2-3 days. Once no bacteria could be observed by microscope examination genomic DNA was extracted from the Drosophila cells using a Wizard Genomic DNA Purification kit using procedures recommended by the manufacture (Promega). Genomic DNA was resuspended on 100 μl 10 mM Tris, pH8.0, 1 mM EDTA and 5 μl was digested for 16 hours with KpnI in a total volume of 20 μl. The DNA digestion reactions were then diluted ten-fold and 1 μl was used as template in a polymerase chain reaction (PCR) containing primers GFP-5Xba (5′-AGTCTAGATATGAGTAAAGGAGA-3′) (SEQ ID No. 21) and GFP-3 Kpn (5′-AGTGGTACCTTATTTGTATAGTT-3′) (SEQ ID No. 22) and using HotStar Tag DNA polymerase as described by the manufacturer (Qiagen). The PCR was performed as follows: Step 1. 95° C. for 15 minutes; Step 2. 5 cycles of 95° C. for 30 seconds, 42° C. for 30 seconds, 70° C. for 1 minute; Step 3. 20 cycles of 95° C. for 30 seconds, 50° C. for 30 seconds, 70° C. for 1 minute; Step 4. 72° C. for 10 minutes.

5 μl of the PCR reaction mixtures were examined by electrophoresis on a 1% agarose gel. The presence of the GFP gene was detected in genomic DNA purified from Drosophila cells co-cultivated with Agrobacteria containing the mutant VirD2 gene encoding a capase-resistant protein (FIG. 8, lane 3). No GFP-specific DNA could be detected in untreated Drosophila cells or in Drosophila cells co-cultivated with Agrobacteria containing a wild type VirD2 gene (lanes 2 and 3).

1. A method of Agrobacterium-mediated gene transformation of a host cell, said Agrobacterium having a VirD2 protein wherein said Agrobacterium has a modified VirD2 protein which is resistant to cleavage by a caspase.

2. The method as claimed in claim 1, wherein said caspase is an animal caspase.

3. The method as claimed in claim 1, wherein said caspase is a plant caspase.

4. The method as claimed in claim 1, wherein said VirD2 protein has at least one of the cleavage sites TATD (SEQ ID NO: 1), GEQD (SEQ ID NO: 2), PVTD (SEQ ID NO: 3) VNLD (SEQ ID NO: 4), ASLD (SEQ ID NO: 5) and DEVD (SEQ ID NO: 6) modified by replacement of the D residue with an alternative amino acid.

5. The method as claimed in claim 4, wherein said VirD2 protein has at least one of the cleavage sites PVTD (SEQ ID NO: 3), VNLD (SEQ ID NO: 4), ASLD (SEQ ID NO: 5) and DEVD (SEQ ID NO: 6) modified by replacement of the D residue with an alternative amino acid.

6. The method as claimed in claim 1, wherein said VirD2 protein has at least one of the cleavage sites TATD (SEQ ID NO: 1) and GEQD (SEQ ID NO: 2) is modified by replacement of the D residue with an alternative amino acid.

7. The method of claim 4 wherein the replacement amino acid is alanine.

8. The method as claimed in claim 1 wherein said host cell is a plant cell.

9. The method as claimed in claim 8 wherein said plant cell is from a monocotyledonous plant.

10. The method as claimed in claim 9 wherein said plant cell is from a cereal.

11. An Agrobacterium vector which expresses a VirD2 protein which is resistant to cleavage by caspases.

12. The Agrobacterium vector as claimed in claim 11 which also encodes a protein of interest.

13. (canceled)

14. (canceled)

15. A VirD2 protein modified by replacement of the D residue of a cleavage site PVTD (SEQ ID NO: 3), VNLD (SEQ ID NO: 4), ASLD SEQ ID NO: 5) or DEVD (SEQ ID NO: 6) with an alternative amino acid.

16. The VirD2 protein as claimed in claim 15 wherein two or more of said cleavage sites are modified by replacement of the D residue with an alternative amino acid.

17. The VirD2 protein as claimed in claim 15 wherein the protein is further modified by replacement of the D residue of the cleavage site TATD (SEQ ID NO: 1) or GEQD (SEQ ID NO: 2) with an alternative amino acid.

18. The VirD2 protein as claimed in claim 15 wherein the alternative amino acid is alanine.

19. The VirD2 protein as claimed in claim 15 that is resistant to cleavage by a caspase.

20. (canceled)

21. A polynucleotide which encodes the modified VirD2 protein as claimed in claim 15.

22. A method of producing a protein of interest, said method comprising:
providing an Agrobacterium vector encoding a VirD2 protein modified to have at least one aspartic acid (D) residue replaced by an alternative amino acid so that the modified VirD2 protein is resistant to cleavage by a caspase, and further comprising a gene encoding a protein of interest within the T-DNA thereof;
infecting a host cell with said Agrobacterium under conditions suitable to allow stable integration of the gene encoding the protein of interest into the genome of said host cell; and
cultivating said host cell or progeny thereof under conditions suitable to allow expression of said protein of interest.


SEQUENCE LISTING (28 sequences)

SEQ ID NO: 1 - protein, 4 aa, Agrobacterium sp.
TATD

SEQ ID NO: 2 - protein, 4 aa, Agrobacterium sp.
GEQD

SEQ ID NO: 3 - protein, 4 aa, Agrobacterium sp.
PVTD

SEQ ID NO: 4 - protein, 4 aa, Agrobacterium sp.
VNLD

SEQ ID NO: 5 - protein, 4 aa, Agrobacterium sp.
ASLD

SEQ ID NO: 6 - protein, 4 aa, Agrobacterium sp.
DEVD

SEQ ID NO: 7 - protein, 60 aa, Agrobacterium sp.
VGPQANAGEQDGSSGPLVRQAGTSRPSPPTATTRASTATDSLSATAHLQQRRGVLSKRPR

SEQ ID NO: 8 - protein, 60 aa, Agrobacterium sp.
VGPQANAGEQAGSSGPLVRQAGTSRPSPPTATTRASTATASLSATAHLQQRRGVLSKRPR

SEQ ID NO: 9 - protein, 4 aa, Agrobacterium sp.
PVTA

SEQ ID NO: 10 - DNA, 19 bp, Artificial
  Chemically synthesised VirD2Ct (D371A) primer
ctactgccagcctgttcgc

SEQ ID NO: 11 - DNA, 18 bp, Artificial
  Chemically synthesised VirD2Ct (D400A) primer
gacaatgaagcggttgcg

SEQ ID NO: 12 - DNA, 21 bp, Artificial
  Chemically synthesised TC178 primer
atcattggcgctcgttacttc

SEQ ID NO: 13 - DNA, 21 bp, Artificial
  Chemically synthesised TC243 primer
cttgtacatagccacatgagc

SEQ ID NO: 14 - protein, 9 aa, Agrobacterium sp.
IIGARYFNK

SEQ ID NO: 15 - protein, 7 aa, Agrobacterium sp.
AHVAMYK

SEQ ID NO: 16 - DNA, 50 bp, Artificial
  Chemically synthesised TC243attB1 primer
ggggacaagtttgtacaaaaaagcaggctcttgtacatagccacatgagc

SEQ ID NO: 17 - DNA, 50 bp, Artificial
  Chemically synthesised TC178attB2 primer
ggggaccactttgtacaagaaagctgggtatcattggcgctcgttacttc

SEQ ID NO: 18 - DNA, 210 bp, Agrobacterium sp.
atcattggcgctcgttacttcaataaaggcctacttgccaacaatccaaatcttaacatttcaatgaattctgctagagataccgatggacatggaactcacacttcttctacagctgcgggaagttatgtcgagggtgcatcttattttggctatgccactggcactgctataggcatagcaccaaaggctcatgtggctatgtacaag

SEQ ID NO: 19 - DNA, 50 bp, Artificial
  Chemically synthesised TC178attB1 primer
ggggacaagtttgtacaaaaaagcaggctatcattggcgctcgttacttc

SEQ ID NO: 20 - DNA, 50 bp, Artificial
  Chemically synthesised TC243attB2 primer
ggggaccactttgtacaagaaagctgggtcttgtacatagccacatgagc

SEQ ID NO: 21 - DNA, 23 bp, Artificial
  Chemically synthesised GFP-5Xba primer
agtctagatatgagtaaaggaga

SEQ ID NO: 22 - DNA, 23 bp, Artificial
  Chemically synthesised GFP-3Kpn primer
agtggtaccttatttgtatagtt

SEQ ID NO: 23 - protein, 4 aa, Artificial Sequence
  Synthetic peptide
TATX

SEQ ID NO: 24 - protein, 4 aa, Artificial Sequence
  Synthetic peptide
GEQX

SEQ ID NO: 25 - protein, 4 aa, Artificial Sequence
  Synthetic peptide
PVTX

SEQ ID NO: 26 - protein, 4 aa, Artificial Sequence
  Synthetic peptide
VNLX

SEQ ID NO: 27 - protein, 4 aa, Artificial Sequence
  Synthetic peptide
ASLX

SEQ ID NO: 28 - protein, 4 aa, Artificial Sequence
  Synthetic peptide
DEVX